(19) United States
(12) Patent Application Publication
(10) Pub. No.: US 2010/0035843 A1
(43) Pub. Date: Feb. 11, 2010

(54) METHODS FOR ATTENUATING ALLERGEN-INDUCED AIRWAY HYPERREACTIVITY USING CD1D DEPENDENT ANTAGONISTS

(75) Inventors: S. Brian Wilson, Lexington, MA (US); Omid Akbari, Brookline, MA (US)

Correspondence Address: DAVID S. RESNICK, NIXON PEABODY LLP, 100 SUMMER STREET, BOSTON, MA, 02110-2131, US

(73) Assignees: THE GENERAL HOSPITAL CORPORATION, Boston, MA (US); CHILDREN'S MEDICAL CENTER CORPORATION, Boston, MA (US)

(21) Appl. No.: 12/525,328

(22) PCT Filed: Feb. 12, 2008
(86) PCT No.: PCT/US08/53688

§ 371 Date: Jul. 31, 2009

Related U.S. Application Data
(60) Provisional application No. 60/900,854, filed on Feb. 12, 2007.

(51) Int. Cl. A61K 31/66

(52) U.S. Cl. 514/121

(57) ABSTRACT

The present invention is directed to a method of inhibiting allergen-induced airway CD1d activation by administering a composition containing a moiety that blocks CD1d activation. Methods of the invention are useful for treatment and prevention of air-way hyperactivity caused by an allergen, and results in the attenuation of CD1d-restricted immune responses, including treatment of hay fever and asthma are due to air-way hyperactivity, and for systemic administration to attenuate ongoing immune responses. Preferably, these compositions are in a form intended for administration via nasal passages or directly inhaled to the air-ways.

### CROSS REFERENCE TO RELATED APPLICATIONS

This application claims benefit under 35 U.S.C. §119(e) of U.S. provisional application No. 60/900,854 filed Feb. 12, 2007, the contents of which are incorporated herein by reference in its entirety.

### GOVERNMENT SUPPORT

This invention was made with Government support under R01 A145051, K11 DK02345, and R01 CA74886 awarded by National Institutes of Health. The Government has certain rights in the invention.

### FIELD OF THE INVENTION

The present invention is directed to a method of attenuating and/or inhibiting allergen-induced airway hyperreactivity. Preferably, the attenuated reaction is an immune response associated with CD1d-dependent activation. The attenuation does not inhibit the OVA-specific MHC class II-dependent Th2 responses.

### BACKGROUND OF THE INVENTION

Allergies are caused by an over-sensitive immune response. The immune system normally protects the body against harmful substances such as bacteria and viruses. Allergy symptoms occur when the immune system reacts to substances (allergens) that are generally harmless and in most people do not cause an immune response.

Airway hyperreactivity in response to various allergens is a significant cause of illness resulting. Airway hypersensitivity causes swelling of the airways when an over-sensitive immune system in the airways becomes exposed to an allergen. Airway hyperreactivity causes allergic rhinitis which is a collection of symptoms, predominantly in the nose and eyes, caused by airborne particles of dust, dander, or plant pollens in people who are allergic to these substances. Allergic rhinitis can develop into asthma or be associated with asthma. Asthma can develop also without preceding rhinitis.

When a person with allergies breathes in an allergen such as pollen or dust, antibodies are produced. When the antibodies are stimulated by pollen and dust, histamine and other chemicals are released. This causes itching, swelling, and mucus production. Symptoms vary from person to person. Very sensitive individuals can also experience hives or other rashes.

For example, hay fever involves an allergic reaction to pollen. A similar reaction occurs with allergy to mold, animal dander, dust, and similar inhaled allergens.

In the United States, about 20 million people have been diagnosed with asthma, and nearly 9 million of them are children. Asthma causes millions of missed work and school days a year and is a common reason for emergency room visits.

Asthma is caused by Th2-driven inflammatory responses, which enhance airway and peripheral blood eosinophilia, induce airway hyperreativity (AHR) and elevate serum IgE6. Conventional class II MHC restricted CD4+ Th2 cells orchestrate the inflammation in asthma (7), by secreting key cytokines, such as IL-4 and IL-13 (8, 9), as well as IL-5 and IL-9 (10). Conventional CD4+ T cells recognize exogenous antigens and initiate allergic inflammation in the lungs and, in mouse models of asthma; elimination of CD4+ cells abrogates the development of AHR 6. Although Th2-driven immune responses are vitally important in the development of asthma (11), a Th2 response however, is not sufficient by itself to induce asthma (11, 12). Th2-biased allergen sensitization can occur independently of asthma, perhaps explaining the fact that only a third of individuals with allergic rhinitis develop asthma (13).

The CD4 marker is expressed not only by conventional CD4+ T cells but also by CD1d restricted natural killer T cells (iNKT cells) that play a crucial role in the development of allergen-induced AHR in several mouse models of asthma (14-18). In these models AHR failed to develop in the absence of iNKT cells even though Th2 responses developed normally. Furthermore, recent studies in humans with asthma suggest that many of CD3+ CD4+ cells in the lungs of patients with persistent asthma are iNKT cells (14). iNKT cells constitute a lymphocyte subpopulation that are abundant in the thymus, spleen, liver and bone marrow and are also present in the lung (14, 15, 19, 20). iNKT cells express surface markers that are characteristic of both natural killer cells and conventional T cells. The largest and best studied population of iNKT cells, recognize glycolipid antigens presented by the non-polymorphic major histocompatibility complex (MHC) class I-like protein CD1d and express an invariant Vα14-Jα18 TCR in mice, or an invariant Vα24-Jα18 TCR in humans (21). When activated, iNKT cells rapidly produce large quantities of several cytokines including IL-4, IL-113, and IFN-γ, which influence subsequent adaptive immune responses and the polarization of conventional αβ-TCR+ T cells (22, 23). Moreover, iNKT cells have been shown to regulate the development of a wide array of autoimmune, antimicrobial, antitumor, antitransplant immune responses, as well as contact sensitivity and asthma (24-26).

The current treatment for asthma include beta-2 sympathomimetics, corticosteroids, parasympatholytics, theophylline, anti-inflammatory agents and anti-allergic agents that are, for instance, administered in the drug treatment of and/or for alleviating asthma, in addition to the still proven means of just avoiding the triggering stimulus.

It would be useful to identify additional agents that directly and specifically attenuate the signaling resulting in airway hyperreactivity. It would also be useful to identify agents that could be used in a prophylactic manner to prevent attacks of airway hyperreactivity, such as asthma, when an individual is at risk of encountering the hyperreactivity-causing environment.

### SUMMARY OF THE INVENTION

We have now discovered that one can specifically attenuate and block CD1d activation in the airways and thus prevent or treat airway hyperreactivity. This can be done by administering compositions that attenuate CD1d-restricted NK T cell responses. Such compositions, when administered in pharmaceutically acceptable carriers, include compounds that act as antagonists by binding CD1d and inhibiting activation of CD1d-restricted NK T cells, compounds that block CD1d-specific receptors on NK T cells, and decoys, mimics and the like. Preferably such compositions are administered directly into the airways using, for example aerosols.

We have discovered that iNKT cells are required for the development of AHR. DPPE-PEG inhibits cytokine production such as IL-4 and IFN-γ by iNKT cells and the development of AHR, but it has no significant effect on antigen-specific Th2 cells. The inhibition of iNKT cell activation is not through the inhibition of the OVA-specific MHC class II-dependent Th2 responses. Compositions that attenuate CD1d-restricted NK T cell responses have no effect on the OVA-specific MHC class II-dependent Th2 responses. In one embodiment, such compositions are administered by inhalation to the lungs.

One preferred group of compositions include phospholipids that bind CD1d without activating NK T cells. Preferred phospholipids include 1,2-Dipalmitoyl-sn-Glycero-3-Phosphoethanolamine (DPPE) and 1,2-Dipalmitoyl-sn-Glycero-3-Phosphoethanolamine-N-[Poly(ethylene glycol)] (DPPE-PEG). Polyethylene glycol (PEG) comes in varying molecular weight and any of the known molecular weight PEG can be used. The numbers that are often included in the names of PEGs indicate their average molecular weight, e.g. a PEG with n=80 would have an average molecular weight of approximately 3500 Daltons and would be labeled PEG 3500. According to the methods of the present invention, any molecular weight for PEG is suitable. For example, we have successfully used at least PEG2000, PEG3000, and PEG3500.

Another preferred group of compositions includes glycolipid antagonists that bind CD1d without activating NK T cells. Preferred glycolipids include ceramide, αMan Cer, and βGal Cer.

Another preferred group of compositions includes phosphatidyl inositol.

We have now discovered that this method of attenuating CD1d activation can be used for treatment of conditions associated with activation of CD1d-restricted NK T cells to treat allergen-induces airway hyperactivity, such as asthma and allergic rhinitis.

A preferred embodiment of the invention is directed to the methods of treatment of asthma.

Another embodiment of the invention is directed to prophylactic methods to prevent an airway hyperreactivity before one encounters the irritant. For example, methods for preventing an asthma attack using the compositions that attenuate CD1d activation by using such an agent or a combination thereof before exposure to an airborne airway irritant.

In one preferred embodiment, the methods of the invention use direct delivery to airways, for example, in inhaled formulation. For the purposes of inhalation, there are available a large number of appliances which can be used to generate aerosols of optimal particle size and administer them using an inhalation technique which is as appropriate as possible for the patient. In addition to using attachments (spacers and expanders) and pear-shaped containers (e.g. NEBULATOR®. and VOLUMATIC®), and also automatic spray puff releasers (AUTOHALER®) for metered aerosols, a number of technical solutions are available, particularly in the case of the powder inhalers (e.g. DISKHALER®, ROTADISK®, TURBOHALER® or the inhaler described in European patent application 0 505 321), which technical solutions can be used to achieve optimal administration of the active compound in accordance with the methods of the invention.

Other embodiments of the invention provide systemic administration of compositions to attenuate ongoing CD1d-restricted immune responses. Systemic therapy can be used in any individual for which activation of NKT cells would be adverse.

Accordingly, in one embodiment, the invention provides a method for treatment and/or prevention of airway hyperreactivity in an individual in need thereof comprising administering to the individual a composition comprising (a) an agent that attenuates CD1d-restricted NK T cells responses, wherein the agent is selected from the group consisting of antagonists that bind CD1d and inhibit activation of CD1d-restricted NK T cells, agents that block CD1d-specific receptors and CD1d decoys and mimics; and (b) a pharmaceutically acceptable carrier or diluent.

In one embodiment, the composition is administered in a pharmaceutically acceptable carrier for inhalable administration.

In one embodiment, the agent is a phospholipid that binds CD1d without activating NK T cells.

In one embodiment, the phospholipid is selected from the group consisting of 1,2-Dipalmitoyl-sn-Glycero-3-Phosphoethanolamine (DPPE), 1,2-Dipalmitoyl-sn-Glycero-3-Phosphoethanolamine-N-[Poly(ethylene glycol) 2000] (DPPE-PEG), and phophotidyl inositol.

In one embodiment, the agent is a glycolipid.

In one embodiment, the glycolipid is selected from the group consisting of ceramide, αMan Cer, and βGal Cer.

In one embodiment, the airway hyperreactivity is associated with asthma.

In another embodiment, the composition is administered systemically.

The invention also provides a method of preventing or reducing a CD1d-restricted NK T cells responses associated in an airway hyperreactivity, comprising administering to an individual in need thereof prior to exposure to an irritant a composition that comprises an agent that attenuates CD1d-restricted NK T cells responses, wherein the agent is selected from the group consisting of antagonists that bind CD1d and inhibit activation of CD1d-restricted NK T cells, agents that block CD1d-specific receptors on NK T cells, and decoys and mimics.

In one embodiment, the irritant is selected from the group consisting of poison ivy, poison oak, poison sumac, insecticides, dye intermediates, resins, detergents, antioxidants, rubber, latex, metals, fragrances, chemicals, cosmetics, textiles, plastics, pollen, therapeutic agents, skin cleansers, industrial cleaning agents, alkalis, acids, oils, organic solvents, oxidizing agents, reducing agents, plant matter, and animal matter.

In one embodiment, the composition is administered by inhalable system.

In another embodiment, the composition is administered by systemic administration.

### BRIEF DESCRIPTION OF THE DRAWINGS

FIGS. 1A-1C show that DPPE-PEG inhibits α-GalCer-dependent activation of iNKT cells in vitro.

FIGS. 1A and 1B show splenocyte cells (5×10⁶/ml) pooled from BALB/cJ mice (n=4) that were cultured in anti-CD3 mAb (2 μg/ml)-precoated 24-well plates in complete RPMI±increasing concentrations of DPPE-PEG350, or that were cultured in 24-well plates in complete RPMI±α-GalCer (100 ng/ml)±increasing concentrations of DPPE-PEG350. After 48 h, supernatants were gathered and examined for IL-4 and INF-γ by ELISA. ELISA data are representative of three separate experiments each and are shown as mean±SD for triplicate samples.

FIG. 1C shows DN32 NKT hybridoma cells that were incubated with 200 μg/ml of DPPE-PEG350 for 4 hours and cultured with increasing concentrations of α-GalCer for 48 h. Culture supernatants were collected and IL-2 expression expression analyzed with IL-2 dependant cell line CTLL-2. Data are means±SD of triplicate cultures.

FIGS. 2A-2D show that AHR is inhibited by the administration of DPPE.

FIG. 2A shows a cohort of 5 BALB/c mice that were immunized with OVA i.p. on day 0, followed by intranasal (i.n.) OVA challenges on days 9, 10 and 11. DPPE (250 μg) was injected i.v. on day 8 and AHR was measured on day 12.

FIG. 2B shows that DPPE prevents AHR and eosinophilic airway inflammation. Methacholine-induced AHR was measured. Administration of DPPE completely inhibited AHR in OVA-immunized mice (measured by enhanced pause, Penh). Data are the mean±SEM Penh, representative of three experiments.

FIG. 2C shows an invasive measurement of airway resistance that was performed in BALB c mice that received DPPE compared to a PBS treated group. AHR was assessed by changes in airway resistance (RL, cmH₂Oper ml per s) in response to methacholine in anesthetized, tracheostomized, intubated and mechanically ventilated mice. Data represent the mean±S.E.M. of 4 mice per group.

FIG. 2D shows that the increased cell number in the BAL fluid of OVA-immunized mice was almost completely abrogated by DPPE. BAL fluid from the mice was analyzed 3 h after airway measurements, shown as the number of cells per ml of BAL fluid. LYM, lymphocyte; EOS, eosinophils; MO, monocyte; NEU, neutrophils.

FIG. 3 shows that DPPE does not inhibit the development of OVA specific Th2 responses. Bronchial lymph nodes were removed from Peg treated or PBS treated mice and restimulated with 100 μg of OVA in vitro. After 72 hrs supernatants were collected and cytokine production analyzed by ELISA. ELISA data are representative of three separate experiments (n=4) and are shown as mean±SD for triplicate samples.

FIG. 4 demonstrates a simplified scheme of antagonistic activities of DPPE-PEG and how it blocks α-GalCer dependant iNKT cell activation.

### DETAILED DESCRIPTION OF THE INVENTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless otherwise stated, the present invention can be performed using standard procedures, as described, for example Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol 57, Jennie P. Mather and David Barnes editors, Academic Press, 1st edition, 1998) which are all incorporated by reference herein in their entireties.

The present invention is directed to methods for treatment and/or prevention of airway hyperreactivity, such as allergic rhinitis and asthma in an individual in need thereof comprising administering to the individual a composition comprising (a) an agent that attenuates CD1d-restricted iNK T cells responses, wherein the agent is selected from the group consisting of antagonists that bind CD1d and inhibit activation of CD1d-restricted iNK T cells, agents that block CD1d-specific receptors and CD1d decoys and mimics; and (b) a pharmaceutically acceptable carrier or diluent. The method does not prevent the development of ovalbumin-specific Th2 responses.

In one embodiment, the airway hyperreactivity is associated with asthma.

Asthma is a major public health problem that has increased markedly in prevalence in the past two decades (1). The prevalence of asthma continues to increase and its optimal treatment remains a significant therapeutic challenge. Recently, CD1d-restricted iNKT cells were found to play a critical role in the induction of airway hyperreactivity (AHR) in rodents and are associated with active asthma in humans. Moreover, studies have demonstrated the ability of phosphatidylethanolamine and DPPE-PEG to bind to CD1d and prevent iNKT cell activation (2-5).

We tested whether iNKT cell-targeted therapy could be used to treat allergen-induced airway hyperreactivity disease (AHR). Mice sensitized with ovalbumin were treated with a CD1d-binding lipid antagonist, DPPE-PEG. This mouse model is commonly used as an asthma model and the results seen in this model can be readily extrapolated to human treatment and clinical trials. Naturally the amount of the agent must be extrapolated to human scale from a mouse scale. However, this kind of scale-up is routine in the art and can be done using, for example, body weight.

We showed that a single dose of DPPE-PEG prevented the development of AHR and pulmonary infiltrates after ovalbumin challenge, but not the development of ovalbumin-specific Th2 responses. Because iNKT cells play a critical role in the development of AHR, the inhibition of iNKT activation by DPPE-PEG showed a role for iNKT cells in AHD and suggests a novel therapy for iNKT cell-mediated diseases such as asthma.

To examine more clearly the role of iNKT cells in the development of allergic disease and asthma, and to determine if iNKT cells might serve as a target for asthma-specific therapy, we evaluated the ability of a CD1d-binding antagonist, DPPE-PEG, to interfere with CD1d presentation and the activation of iNKT cells in AHR.

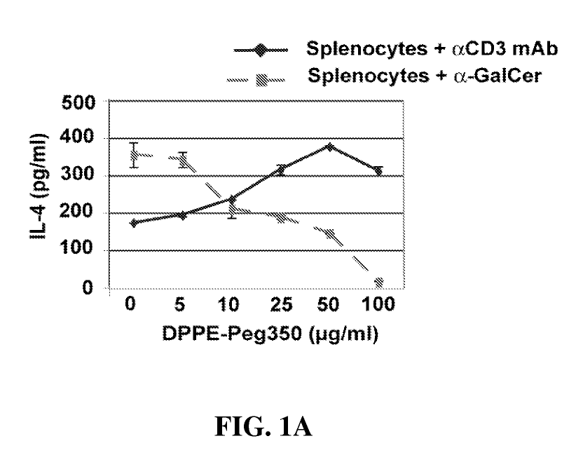

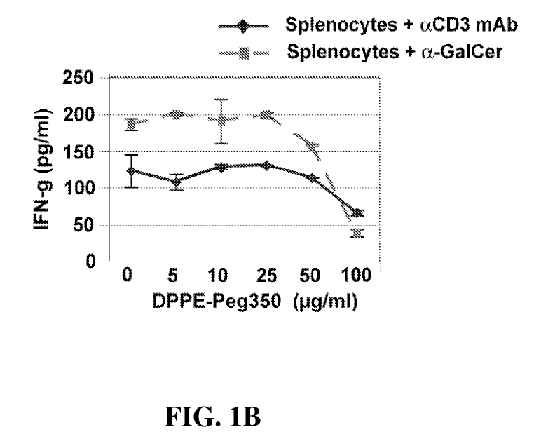

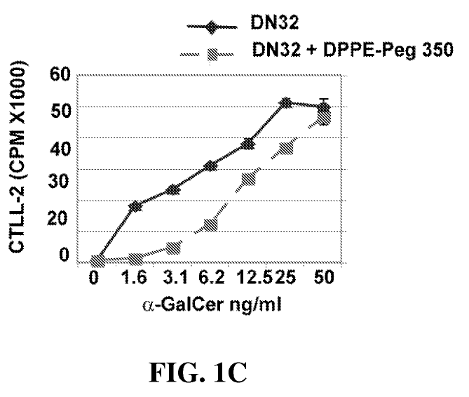

First we determined if the activation of iNKT cells by α-GalCer could specifically be inhibited by DPPE-PEG, by stimulating splenocytes with α-GalCer (100 ng/ml) or plate-bound anti-CD3. The addition of increasing concentrations of DPPE-PEG inhibited secretion of IL-4, and to a lesser extent, IFN-γ by α-GalCer-stimulated iNKT cells. Treatment with DPPE-PEG350 preferentially inhibited iNKT cells cytokine release when compare to anti-CD3 activated T cells (FIGS. 1A and B). Importantly, the α-GalCer-stimulated cytokine secretion by iNKT cells was significantly inhibited at concentrations of DPPE-PEG that had no or minimal effect on cytokine production by anti-CD3 activated T cells.

We also tested the ability of DPPE-PEG350 to block the iNKT cell hybridoma line DN32, activation by α-GalCer. DN32 cells were loaded with 200 μg/ml of DPPE-PEG, and stimulated with titrating dose of α-GalCer. Treatment with DPPE-PEG350 inhibited IL-2 release from DN32 iNKT cells, particularly at suboptimal concentrations of α-GalCer (FIG. 1C).

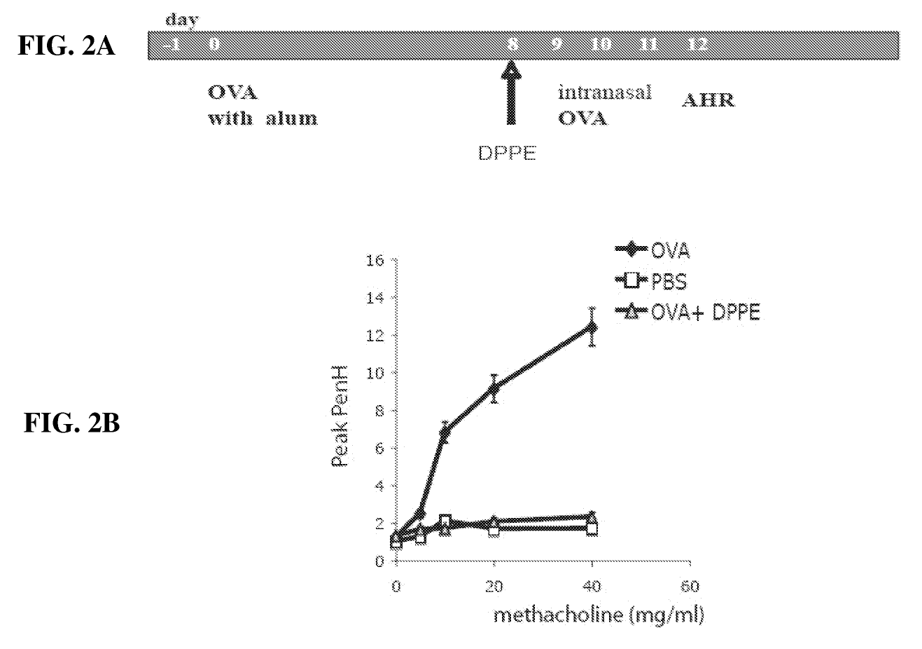

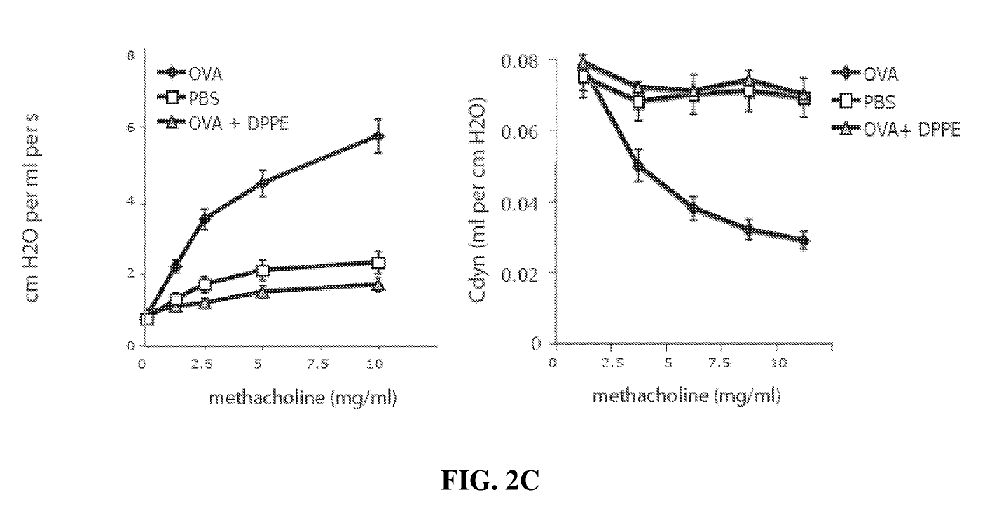

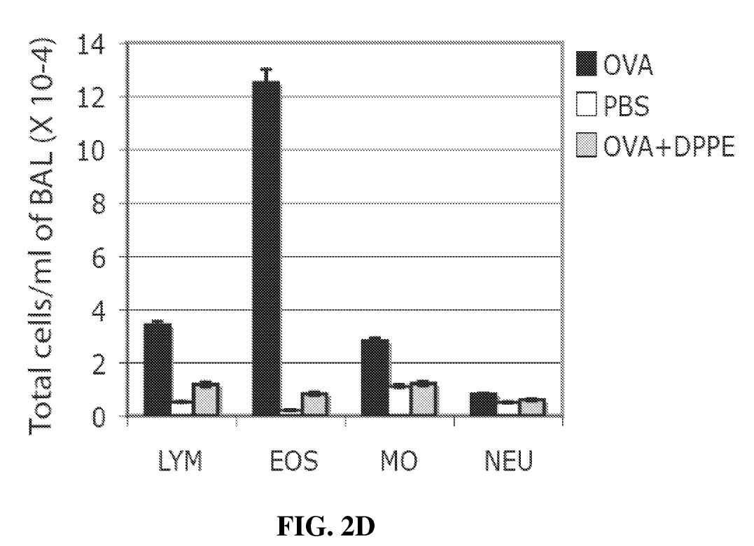

To determine whether DPPE-PEG350 could be used to treat the Th2 cell-mediated immune response of AHR, the drug was tested in an OVA-induced model of AHR, previously demonstrated to require the presence of iNKT cells (15, 16). In this model, an asthma-like phenotype with AHR, eosinophilic airway inflammation and mucus hypersecretion is induced by systemic sensitization with OVA followed by intrapulmonary challenge with OVA (FIG. 2A). Following sensitization, injection of a single dose of DPPE-PEG350 (250 μg i.v.), 1 day before the OVA challenge, prevented the development of AHR in BALB/c mice (FIG. 2B). AHR was measured by PenH in a whole-body plethysmograph (FIG. 2B), and was confirmed by direct measurement of airway resistance (RL) and dynamic compliance (Cdyn; a measure of the elasticity of the lung) in anesthetized, tracheostomized, intubated, and mechanically ventilated BALB/c mice (FIG. 2C). In addition, treatment with DPPE-PEG350 effectively in inhibited the development of eosinophilic airway inflammation in OVA-immunized BALB/c mice, thus significantly reducing the number of eosinophils, lymohocytes, and macrophages in the bronchoalveolar lavage (BAL) fluid as compared with the OVA-immunized control group (FIG. 2D). Moreover, histological examination of H&E-stained lung sections isolated from OVA-immunized mice revealed no significant differences between saline (negative) control and DPPE-PEG treated mice (data not shown). In contrast, OVA-sensitized and challenged mice that did not receive DPPE-PEG developed intense cellular infiltrates. Thus, we conclude that DPPE-PEG prevents OVA-induced AHR.

Accordingly, the invention provides a novel prevention method of asthma, wherein the preventive agent can be administered prior to the exposure to the allergen and will thus prevent an asthma attack.

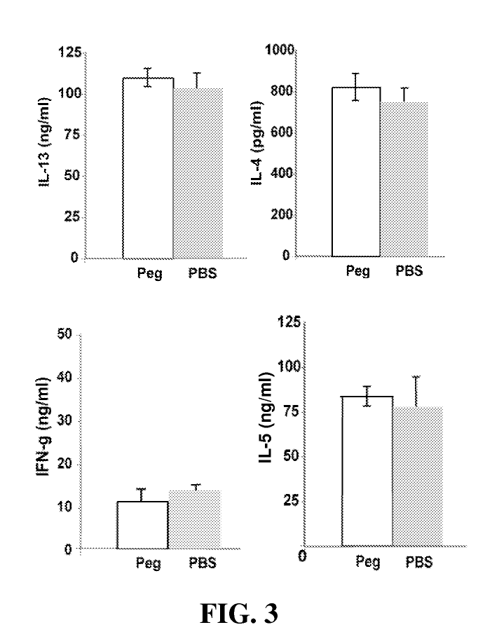

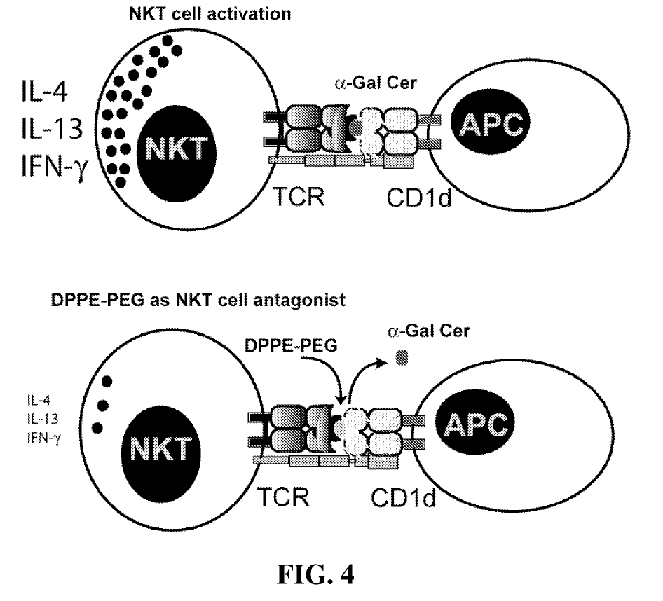

In this model of AHR the response is dependent on CD4+ OVA-specific Th2 cells. Hence, ex vivo challenge of lymphocytes from sensitized animals secrete large amounts of IL-4, IL-5 and IL-13 in response to antigen. Since treatment with DPPE-PEG350 inhibited the development of AHR, we next asked if administration of the CD1d-dependent antagonist prior to airway challenge altered systemic OVA-specific Th2 responses. Bronchial lymph node cells were isolated from OVA sensitized mice treated with either DPPE-PEG350 or PBS, and restimulated in vitro with 100 μg/ml of ovalbumin a day after induction of AHR. No significant difference in the levels of IL-4, IL-5 or IL-13 secretion to OVA recall was seen when comparing the DPPE-PEG350 treated group with PBS treated group (FIG. 3). No OVA-specific cytokine secretion was seen if the animals had not been sensitized with OVA on day 0 (data not shown). Hence, the iNKT-dependent development of AHR, but not OVA-specific MHC class II-dependent Th2 responses, can be inhibited by DPPE-PEG.

Asthma is a complex disorder characterized by mucous hypersecretion, AHR and cellular infiltrates dominated by eosinophils and CD4+ lymphocytes (27). The large numbers of infiltrating CD4+ T cells are biased to secrete the Th2-related cytokines IL-4 and IL-13. Recently, it was reported that in humans a significant fraction of these T cells were CD4+ iNKT cells (14), although the degree of infiltration by iNKT cells has been questioned (28). Nevertheless, there is strong support for the notion that allergen-induced AHR in mice, a cardinal feature of asthma, fails to develop in the absence of iNKT cells (15, 16). The failure of iNKT cell-deficient mice to develop AHR is not due to a general inability of these mice to produce Th2 responses because iNKT cell-deficient mice that are immunized subcutaneously at non-mucosal sites produce normal antigen-specific Th2 responses (15). Moreover, reconstitution of these mice with iNKT cells restores the capacity of these mice to develop AHR. Furthermore, direct activation of pulmonary iNKT cells by the administration of α-GalCer induces AHR and airway inflammation in the complete absence of class II MHC restricted T cells (29). Thus, pulmonary iNKT cells alone can support the development of AHR

Despite the fact that the strong Th2 biases of the immune responses in asthma and allergy are relatively well characterized, the specific mechanisms whereby iNKT cells contribute to the development of AHR are poorly understood. Interestingly, iNKT cells are also required for the development of experimental contact hypersensitivity (CHS), a prototypic model for allergic contact dermatitis (3, 30). In the CHS model, Nieuwenhuis et al. found that dermatitis was diminished in iNKT deficient animals and could be blocked by treatment with DPPE-PEG. PEG modified lipid and related family members were shown to bind CD1d, and in the case of unmodified PE, function as weak agonists for rare iNKT cell hybridomas (3, 31).

We hypothesized that modification of the polar head group would sterically hinder recognition by CD1d-restricted T cells and yet accommodate binding to CD1d via the lipid tails. Additionally, PEG-modified lipids, and DPPE-PEG in particular, specifically target DC, a critical partner and important target of iNKT cell immunoregulatory function (32, 33).

To evaluate whether the pharmacologic antagonism of iNKT cells could inhibit AHR, BALB/c mice were sensitized with OVA and then one day prior to intranasal challenge with OVA, mice were treated with vehicle or DPPE-PEG i.v. AHR, as measured by PenH, airway resistance dynamic compliance, as well as airway inflammation, as measured in BAL, and by immunohistochemistry, was significantly inhibited in mice treated with DPPE-PEG. Hence, it seems reasonable to suggest that DPPE-PEG specifically inhibits iNKT cell activation. This conclusion is supported by the literature and the fact that a dose of DPPE-PEG sufficient to block α-GalCer-dependent iNKT activation in vitro did not affect anti-CD3 stimulated T cell cytokine secretion or the ova-specific secretion of, IL-4, IL-5, IL-13 and IFN-γ after in vitro re-challenge of sensitized and DPPE-PEG treated animals. Since iNKT cells have been found in the lungs of asthma patients, and since phosphatidylethanolamine derived from cypress pollen is recognized by iNKT cells in cypress allergic individuals (4, 34), based on our experiments, we suggest that iNKT cell-based therapies using DPPE-PEG are effective in treating patients with allergic asthma and allergen-induced rhinitis (4, 14, 34).

It was previously reported that inhibiting CD1d reaction in skin, various skin disorders can be treated (U.S. Patent Application Publication No. 20020165170).

One can readily analyze, using routine methods, compounds that inhibit activation of CD1d by determining whether a compound inhibits activation of CD1d by looking at activation of CD1d-restricted NK T cells in vitro using standard assays such as described herein. For example, the proliferation of NK T cells is indicative of their activation by binding to CD1d-expressing cells. Proliferation can be measured, for example, by determining the incorporation of [³H] thymidine into Vα14 NK T cells (Kawano et al., Science 278:1626-29 (1997)). Other in vitro assays include the induction of cytokine production.

CD1d-specific antagonists useful according to the methods of the present invention include any antagonist that binds CD1d and inhibits activation of NK T cells. The binding of an antagonist to CD1d can be determined in vitro using standard assays. For example, surface plasmon resonance (Naidenko et al., J. Exp. Med. 190:1069-79 (1999)). Molecules known to bind CD1d include antibodies, phospholipids and glycolipids, including highly glycosylated sphingolipids (gangliosides) (Kawano et al., Science 278: 1626-9 (1997); Naidenko et al., J. Exp. Med. 190:1069-79 (1999); Briken et al., Sem. Immunol. 12: 517-25 (2000); Kronenberg et al., Proc. Natl. Acad. Sci. USA 98: 2950-52 (2001)).

In one embodiment, an agent that attenuates CD1d-restricted NK T cells responses is a phospholipid that binds CD1d without activating NK T cells.

Phospholipids that bind CD1d without activating NK T cells include 1,2-Dipalmitoyl-sn-Glycero-3-Phosphoethanolamine (DPPE) (Naidenko et al., 1999). The phospholipid may be coupled to a conjugate, such as other lipids coupled to a carrier such as biotin or a poly(alkaline oxide), for example polyethylene glycol (PEG). Polymeric substances such as dextran, polyvinyl pyrrolidones, polysaccharides, starches, polyvinyl alcohols, polyacrvyl amides or other similar polymers can be used. Polyethylene glycol (PEG) as the poly(alkylene oxide) is preferred. The poly(alkylene oxides) can include monomethoxy polyethylene glycol, polypropylene glycol, block copolymers of polyethylene glycol and polypropylene glycol and the like. The polymers can also be distally capped with C1-4 alkyls instead of monomethoxy groups.

Accordingly, the phospholipid can be selected from the group consisting of 1,2-Dipalmitoyl-sn-Glycero-3-Phosphoethanolamine (DPPE), 1,2-Dipalmitoyl-sn-Glycero-3-Phosphoethanolamine-N-[Poly(ethylene glycol) 2000] (DPPE-PEG), and phophotidyl inositol.

Other preferred phospholipids include phosphotidyl inositol.

In another embodiment, an agent that attenuates CD1d-restricted NK T cells responses is a glycolipid.

Glycolipids that bind to CD1d share a common motif consisting of a hydrophobic portion composed of a branched or dual alkyl chain moiety with a covalently linked hydrophilic cap formed by a polar or charged group of the lipid of associated carbohydrates (Briken et al., 2000). The prototypical glycolipid antigen presented by CD1d is α-galactosylceramide (Kawano et al., 1997). Thus, D1d antagonists can include glycolipids such as monoglycosylated ceramides and diglycosylated ceramides (Kawano et al., 1997).

Examples of monoglycosylated ceramides and diglycosylated ceramides which bind CD1d but do not activate NK T cells can include ceramides with inner sugar groups at the β-anomer position (such as Galα1-4Glcβ1-1′Cer), an axial configuration of the 2-hydroxyl group (such as α-ManCer), derivatives lacking the 3- and 4-hydroxyl groups on the phytosphingosine of α-GalCer (such as 3,4-deoxy α-GalCer), and ceramides with fatty acyl chain with less than C26, and a sphingosine base less than C18. Glycolipid antagonists can also be coupled to a conjugate such as biotin or a poly(alkylene oxide), for example polyethylene glycol (PEG).

Other CD1d glycolipid antagonists include highly glycosylated sphingolipids, also known as gangliosides. Ganglioside antagonists include GM1 and GD1a (Naidenko et al., 1997). The antagonists can be coupled to a conjugate such as a biotin or poly(alkylene oxide).

Preferred examples of CD1d-specific lipid antagonists include but are not limited to: DPPE-PEG, phosphatidyl inositol, ceramide, α-ManCer, β-GalCer, Galα1-4Glcβ1-1′Cer, and 13,4-deoxy α-GalCer, GM1, and GD1a.

Accordingly, the glycolipid can be selected from the group consisting of ceramide, αMan Cer, and βGal Cer.

Other antagonists can include antibodies that specifically bind to CD1d and in doing so prevent CD1d from binding to a CD1d specific receptor. Single chain antibodies and humanized monoclonal antibodies are preferred. Alternatively, one can use molecules that block the CD1d-specific receptor. For example, a molecule. Alternatively, one can modify the lipids that activate CD1d binding by capping the end that bind to CD1d (Kawano et al., Science 278: 1626-9 (1997); Naidenko et al., J. Exp. Med. 190:1069-79 (1999); Briken et al., Sem. Immunol. 12: 517-25 (2000); Kronenberg et al., Proc. Natl. Acad. Sci. USA 98: 2950-52 (2001)).

One can also use decoys that mimic CD1d receptors so that the CD1d molecule does not bind to the receptor.

CD1d blocking molecules are presently preferred.

In one embodiment, the invention also provides a method of preventing or reducing a CD1d-restricted NK T cells responses associated in an airway hyperreactivity, comprising administering to an individual in need thereof prior to exposure to an irritant a composition that comprises an agent that attenuates CD1d-restricted NK T cells responses, wherein the agent is selected from the group consisting of antagonists that bind CD1d and inhibit activation of CD1d-restricted NK T cells, agents that block CD1d-specific receptors on NK T cells, and decoys and mimics.

In one embodiment, the irritant is selected from the group consisting of poison ivy, poison oak, poison sumac, insecticides, dye intermediates, resins, detergents, antioxidants, rubber, latex, metals, fragrances, chemicals, cosmetics, textiles, plastics, pollen, therapeutic agents, skin cleansers, industrial cleaning agents, alkalis, acids, oils, organic solvents, oxidizing agents, reducing agents, plant matter, and animal matter.

Compositions useful according to the methods of the present invention should be physiologically stable at therapeutically effective concentrations. Physiological stable compounds are compounds that do not break down or otherwise become ineffective upon introduction to a patient prior to having a desired effect. Compounds are structurally resistant to catabolism, and thus, physiologically stable, or coupled by electrostatic or covalent bonds to specific reagents to increase physiological stability. Such reagents include amino acids such as arginine, glycine, alanine, asparagine, glutamine, histidine or lysine, nucleic acids including nucleosides or nucleotides, or substituents such as carbohydrates, saccharides and polysaccharides, lipids, fatty acids, proteins, or protein fragments. Useful coupling partners include, for example, glycol such as polyethylene glycol, glucose, glycerol, glycerin and other related substances.

Preferably, the compositions are not substantially toxic, myelotoxic, mutagenic or teratogenic at required dosages. Although side effects may occur, preferably the benefits achieved from their use outweigh disadvantages attributable to adverse side effects.

Prophylactic uses to attenuate air-way hyperactivity resulting from immune responses include, for example, to avoid reactions to air-way irritant that one encounters in occasions, such as leisure activities, work or special when one is going to be in an area where exposure to such a substance is likely, for example, if one is going to be in the woods, gardening, exposed to tobacco smoke etc.

In one embodiment, the composition is administered systemically. In another embodiment, the composition is administered in in a pharmaceutically acceptable carrier for inhalable administration. Direct administration to the airways is preferred, for example in an aerosol format suitable for inhaling the compounds directly into the airways. However, one can also use systemic administration in any individual for which activation of NKT cells would be adverse.

For the treatment of airway hyperreactivity according to the methods of the present invention, the compounds that block CD1d receptor activity are preferably also administered by inhalation, preferably in the form of an aerosol, with the aerosol particles of solid, liquid or mixed composition having a diameter of from 0.5 to 10 μm, advantageously of from 2 to 6 μm.

The aerosol can be produced, for example, using pressure-driven nozzle nebulizers or ultrasonic nebulizers, advantageously, however, using propellant gas-driven metered aerosols or by means of the propellant gas-free use of micronized active compounds from inhalation capsules.

Depending on the inhalation system employed, the administration forms also contain, In addition to the active compounds, the requisite auxiliary substances, for example propellant gases, surface-active substances, emulsifiers, stabilizers, preservatives, aromatizing agents, fillers (e.g. lactose in the case of powder inhalers) and, where appropriate, additional active compounds.

One can also use the method of the present invention to treat reactions that are prolonged even after the initial stimulus is gone. This is a particular problem with non-human animals. These compositions can be used to attenuate, treat air-way irritation in such situations as well.

One preferred embodiment of the invention provides prophylactic treatment to minimize airway hyperreactivity produced as a therapeutic drug. In another embodiment, the composition is used for treatment after the reaction has occurred.

Another embodiment of the invention provides systemic administration of compositions to attenuate ongoing CD1d-restricted immune responses in the airways. This embodiment is preferable for any individual for whom activation of NKT cells would be adverse.

The compositions of the present invention include those suitable systemic administration including oral, nasal, or parenteral administration, all of which may be used as routes of administration using the compounds as described useful according to the present invention. A preferred route of administration is an inhaled aerosol.

Compositions may be administered to the nasal passages as a spray. Arteries of the nasal area provide a rapid and efficient access to the bloodstream and immediate access to the pulmonary system. Access to the gastrointestinal tract, which can also rapidly introduce substances to the blood stream, can be gained using oral enema, or injectable forms of administration. Compositions may be administered as a bolus injection or spray, or administered sequentially over time (episodically) such as every two, four, six or eight hours, every day (QD) or every other day (QOD), or over longer periods of time such as weeks to months. Compositions may also be administered in a timed-release fashion such as by using slow-release resins and other timed or delayed release materials and devices.

Systemic administration of a composition may be by oral, parenteral, sublingual, rectal such as suppository or enteral administration, or by pulmonary absorption. Parenteral administration may be by intravenous injection, subcutaneous injection, intramuscular injection, intra-arterial injection, intrathecal injection, intra peritoneal injection or direct injection or other administration to one or more specific sites. When long term administration by injection is necessary, venous access devices such as medi-ports, in-dwelling catheters, or automatic pumping mechanisms are also preferred wherein direct and immediate access is provided to the arteries in and around the heart and other major organs and organ systems.

Where systemic administration is desired, orally active compositions are preferred as oral administration is a convenient and economical mode of drug delivery. Oral compositions may be poorly absorbed through the gastrointestinal lining. Compounds which are poorly absorbed tend to be highly polar. Preferably, such compositions are designed to reduce or eliminate their polarity. This can be accomplished by known means such as formulating the oral composition with a complimentary reagent which neutralizes its polarity, or by modifying the compound with a neutralizing chemical group. Preferably, the molecular structure is similarly modified to withstand very low pH conditions and resist the enzymes of the gastric mucosa such as by neutralizing an ionic group, by covalently bonding an ionic interaction, or by stabilizing or removing a disulfide bond or other relatively labile bond.

Treatments to the patient may be therapeutic or prophylactic. Therapeutic treatment involves administration of one or more compositions of the invention to a patient suffering from one or more symptoms of the disorder. Relief and even partial relief from one or more symptoms can correspond to an increased life span or simply an increased quality of life. Further, treatments that alleviate a pathological symptom can allow for other treatments to be administered.

The term “compatible”, as used herein, means that the components of the compositions are capable of being commingled with the CD1d blocking agents of the present invention, and with each other, in a manner such that does not substantially impair the desired efficacy.

Doses of the pharmaceutical compositions of the invention will vary depending on the subject and upon the particular route of administration used. Dosages can range from 0.1 to 100,000 μg/kg per day, for example, 1 to 10,000 μg/kg. By way of an example only, an overall dose range of from about, for example, 1 microgram to about 300 micrograms might be used for human use. This dose can be delivered at periodic intervals based upon the composition.

This invention is further illustrated by the following example which should not be construed as limiting. The contents of all references cited throughout this application, as well as the figures and table are incorporated herein by reference.

### EXAMPLES

### Material and Methods

Mice and antigens. Female BALB/cJ mice (6-8 weeks old) were purchased from The Jackson Laboratory (Bar Harbor, Me.). All mice were maintained in a pathogen-free mouse colony at The Children's Hospital of Boston and Massachusetts General Hospital under IACUC approved mouse protocols.

Reagents. Anti-CD3 mAb was purchased from BD PharMingen (San Diego, Calif.). α-Galactosylceramide (α-Gal-cer; KRN7000) was purchased from Axxora LLC (San Diego, Calif.). DPPE-PEG. (1,2-Dipalmitoyl-sn-Glycero-3-Phosphoethanolamine-N-[Methoxy(Polyethylene glycol)-350]) (Ammonium Salt) (PEG350) was purchased from Avanti Polar Lipids, INC (Alabaster, Ala.). We thank A. Bendelac for the DN32.D3 hybridoma cells. To induce AHR, mice were immunized intraperitoneally with 50 μg of LPS free OVA (Worthington Biochemical Corp., NJ) in 2 mg of alum in volume of 0.5 ml. Ten days later mice were lightly anesthetized with methoxoflurane and challenged with i.n. OVA (50 μg) on 3 consecutive days.

Restimulation of lymph node cells in vitro. Cells isolated from lymph nodes of OVA-primed mice were restimulated in vitro (5×105 cells per well in a 96-well plate) with various concentrations of OVA. Supernatants were collected after 4 days and assayed for IL-4 and IFN-γ by ELISA as previously described (15).

Inhibition of in vitro iNKT activation with DPPE-PEG-Splenocytes cells (5×106/ml) pooled from BALB/cJ mice (n=4) were cultured in anti-CD3 mAb (2 μg/ml)-precoated 24-well plates in complete RPMI±increasing concentrations of DPPE-PEG350, or cultured in 24-well plates in complete RPMI±KRN7000 (100 ng/ml)±increasing concentrations of DPPE-PEG350. After 48 h, supernatants were gathered and examined for IL-4 and INF-γ by ELISA as described (35).

Cytokine ELISAs—Cytokine secretion following in vitro challenge with OVA and control peptides were determined by ELISA as described before (15). Briefly, the following monoclonal antibodies R4-6A2 and XMG1.2-biotin for IFN-γ and 11B11 and BVD6-24G2-biotin for IL-4 were used for capture and detection.

Induction of AHR and measurement of airway responsiveness—AHR responses were assessed by methacholine-induced airflow obstruction in conscious mice placed in a whole-body plethysmograph (Buxco Electronics, Troy, N.Y.) as described previously 15. In some experiments AHR was assessed by invasive measurement of airway resistance, in which anesthetized and tracheostomized mice were mechanically ventilated using a modified version of a described method 36. Aerosolized methacholine was administered for 20 breaths in increasing concentrations (1.25, 2.5, 5 and 10 mg/ml of methacholine). We continuously computed RL and Cdyn by fitting flow, volume and pressure to an equation of motion.

Collection and analysis of BAL fluid—Mice were lethally anesthetized with phenobarbital 450 mg/kg i.p. The trachea was cannulated and the lungs were lavaged three times with 0.3 ml of PBS, and the collected fluid was pooled. Cell counts and analysis of the BAL was performed as described (15).

DPPE-PEG inhibits α-GalCer-dependent activation of iNKT cells in vitro.

Phosphatidylethanolamine and DPPE-PEG bind to CD1d and prevent iNKT cell activation (2-5). To determine whether activation of iNKT cells by α-GalCer could specifically be inhibited by DPPE-PEG, splenocytes were incubated with α-GalCer (100 ng/ml) or plate-bound anti-CD3. FIGS. 1A-1C show that DPPE-PEG inhibits α-GalCer-dependent activation of iNKT cells in vitro. FIGS. 1A and 1B show splenocyte cells (5×10⁶/ml) pooled from BALB/cJ mice (n=4) that were cultured in anti-CD3 mAb (2 μg/ml)-precoated 24-well plates in complete RPMI±increasing concentrations of DPPE-PEG350, or that were cultured in 24-well plates in complete RPMI±α-GalCer (100 ng/ml)±increasing concentrations of DPPE-PEG350. After 48 h, supernatants were gathered and examined for IL-4 and INF-γ by ELISA. ELISA data are representative of three separate experiments each and are shown as mean±SD for triplicate samples. FIG. 1C shows DN32 NKT hybridoma cells that were incubated with 200 μg/ml of DPPE-PEG350 for 4 hours and cultured with increasing concentrations of α-GalCer for 48 h. Culture supernatants were collected and IL-2 expression expression analyzed with IL-2 dependant cell line CTLL-2. Data are means±SD of triplicate cultures.

The addition of increasing concentrations of DPPE-PEG inhibited secretion of IL-4, and to a lesser extent, IFN-γ by α-GalCer-stimulated iNKT cells. Treatment with DPPE-PEG350 preferentially inhibited iNKT cells cytokine release when compare to anti-CD3 activated T cells (FIGS. 1A and B). Importantly, the α-GalCer-stimulated cytokine secretion by iNKT cells was significantly inhibited at concentrations of DPPE-PEG that had no or minimal effect on cytokine production by anti-CD3 activated T cells.

We also tested the ability of DPPE-PEG350 to block the iNKT cell hybridoma line DN32, activation by α-GalCer. DN32 cells were loaded with 200 μg/ml of DPPE-PEG, and stimulated with titrating dose of α-GalCer. Treatment with DPPE-PEG350 inhibited IL-2 release from DN32 iNKT cells, particularly at suboptimal concentrations of α-GalCer (FIG. 1C).

DPPE-PEG350 treatment inhibits development of allergic airway hyperreactivity.

To determine whether DPPE-PEG350 could be used to treat the Th2 cell-mediated immune response of AHR, the drug was tested in an OVA-induced model of AHR, previously demonstrated to require the presence of iNKT cells (15). In this model, an asthma-like phenotype with AHR, eosinophilic airway inflammation and mucus hypersecretion is induced by systemic sensitization with OVA followed by intrapulmonary challenge with OVA (FIG. 2A).

FIGS. 2A-2D show that AHR is inhibited by the administration of DPPE. FIG. 2A shows a cohort of 5 BALB/c mice that were immunized with OVA i.p. on day 0, followed by intranasal (i.n.) OVA challenges on days 9, 10 and 11. DPPE (250 μg) was injected i.v. on day 8 and AHR was measured on day 12. FIG. 2B shows that DPPE prevents AHR and eosinophilic airway inflammation. Methacholine-induced AHR was measured. Administration of DPPE completely inhibited AHR in OVA-immunized mice (measured by enhanced pause, Penh). Data are the mean±SEM Penh, representative of three experiments. FIG. 2C shows an invasive measurement of airway resistance that was performed in BALB c mice that received DPPE compared to a PBS treated group. AHR was assessed by changes in airway resistance (RL, cmH₂Oper ml per s) in response to methacholine in anesthetized, tracheostomized, intubated and mechanically ventilated mice. Data represent the mean±S.E.M. of 4 mice per group. FIG. 2D shows that the increased cell number in the BAL fluid of OVA-immunized mice was almost completely abrogated by DPPE. BAL fluid from the mice was analyzed 3 h after airway measurements, shown as the number of cells per ml of BAL fluid. LYM, lymphocyte; EOS, eosinophils; MO, monocyte; NEU, neutrophils. Histological staining of lung tissue shows that DPPE inhibits airway inflammation (data not shown). The lung tissue from an untreated control mouse have normal airway and surrounding parenchyma. Airway mucosa showing low cuboidal cells with minimal intracytoplasmic mucus and absence of peribronchiolar inflammatory infiltrates. The lung tissue from a OVA-treated mouse have numerous inflammatory cells surrounding the airways and streaks of mucus in the lumen. Bronchiolar epithelium showing hyperplastic columnar epithelial cells with abundant intracytoplasmic accumulations of mucous, as well as eosinophils and mononuclear cells in the peribronchial space can be seen. The lung parenchyma of an OVA-sensitized mouse that has received DPPE have minimal mucus production and negligible cellular infiltration. Bronchiolar mucosa consists of low cuboidal epithelium with an absence of peribronchiolar inflammatory infiltrates. All staining is hematoxylin/eosine (H&E) and the magnification is ×400.

Following sensitization, injection of a single dose of DPPE-PEG350 (250 μg i.v.), 1 day before the OVA challenge, completely prevented the development of AHR in BALB/c mice (FIG. 2B). AHR was measured by PenH in a whole-body plethysmograph (FIG. 2B), and was confirmed by direct measurement of airway resistance (RL) and dynamic compliance (Cdyn; a measure of the elasticity of the lung) in anesthetized, tracheostomized, intubated, and mechanically ventilated BALB/c mice (FIG. 2C). In addition, treatment with DPPE-PEG350 very effectively in inhibited the development of eosinophilic airway inflammation in OVA-immunized BALB/c mice, thus significantly reducing the number of eosinophils, lymohocytes, and macrophages in the bronchoalveolar lavage (BAL) fluid as compared with the OVA-immunized control group (FIG. 2D). Moreover, histological examination of H&E-stained lung sections isolated from OVA-immunized mice revealed no significant differences between saline (negative) control and DPPE-PEG treated mice (data not shown). In contrast, OVA-sensitized and challenged mice that did not receive DPPE-PEG developed intense cellular infiltrates. Thus, we conclude that DPPE-PEG prevents OVA-induced AHR.

DPPE-PEG350 is specific for AHR and does not inhibit the development of systemic OVA specific TH2 responses.

In this model of AHR the response is dependent on CD4+ OVA-specific Th2 cells. Hence, ex vivo challenge of lymphocytes from sensitized animals secrete large amounts of IL-4, IL-5 and IL-13 in response to antigen (15, 37).

FIG. 3 shows that DPPE does not inhibit the development of OVA specific Th2 responses. Bronchial lymph nodes were removed from Peg treated or PBS treated mice and restimulated with 100 μg of OVA in vitro. After 72 hrs supernatants were collected and cytokine production analyzed by ELISA. ELISA data are representative of three separate experiments (n=4) and are shown as mean±SD for triplicate samples.

Since treatment with DPPE-PEG350 inhibited the development of AHR, we next asked if administration of the CD1d-dependent antagonist prior to airway challenge altered systemic OVA-specific Th2 responses. Bronchial lymph node cells were isolated from OVA sensitized mice treated with either DPPE-PEG350 or PBS, and restimulated in vitro with 100 μg/ml of ovalbumin a day after induction of AHR. No significant difference in the levels of IL-4, IL-5 or IL-13 secretion to OVA recall was seen when comparing the DPPE-PEG350 treated group with PBS treated group (FIG. 3). No OVA-specific cytokine secretion was seen if the animals had not been sensitized with OVA on day 0 (data not shown). Hence, the iNKT-dependent development of AHR, but not the OVA-specific MHC class II-dependent Th2 responses, can be inhibited by DPPE-PEG.

Our results indicate that DPPE-PEG inhibits cytokine production such as IL-4 and IFN-γ by iNKT cells and the development of AHR, but it has no significant effect on antigen-specific Th2 cells. Thus, our results support the idea that iNKT cells are required for the development of AHR and that an iNKT cell specific antagonist such as DPPE-PEG, can target iNKT cells as a potential therapeutic approach for allergic inflammatory responses and other disorders characterized by inappropriate iNKT cell activation.

The references cited herein and throughout the specification are incorporated herein by reference.

### REFERENCES

1. Forecasted state-specific estimates of self-reported asthma prevalence—United States, 1998. Mmwr. Morbidity and Mortality Weekly Report 47, 1022-5 (1998).
2. Naidenko, O. V. et al. Binding and antigen presentation of ceramide-containing glycolipids by soluble mouse and human CD1d molecules. J Exp Med 190, 1069-80 (1999).
3. Nieuwenhuis, E. E. et al. CD1d and CD1d-restricted iNKT-cells play a pivotal role in contact hypersensitivity. Exp Dermatol 14, 250-8 (2005).
4. Russano, A. M. et al. Recognition of pollen-derived phosphatidyl-ethanolamine by human CD1d-restricted gamma delta T cells. J Allergy Clin Immunol 117, 1178-84 (2006).
5. De Silva, A. D. et al. Lipid protein interactions: the assembly of CD1d1 with cellular phospholipids occurs in the endoplasmic reticulum. J Immunol 168, 723-33 (2002).
6. Wills-Karp, M. Immunologic basis of antigen-induced airway hyperresponsiveness. Annu. Rev. Immunol. 17, 255-81 (1999).
7. Robinson, D. S. et al. Predominant Th2-like bronchoalveolar T-lymphocyte population in atopic asthma. N. Engl. J. Med. 326, 298-304 (1992).
8. Grunig, G. et al. Requirement for IL-13 independently of IL-4 in experimental asthma. Science 282, 2261-3 (1998).
9. Wills-Karp, M. et al. Interleukin-13: central mediator of allergic asthma. Science 282, 2258-61 (1998).
10. Holt, P. G., Macaubas, C., Stumbles, P. A. & Sly, P. D. The role of allergy in the development of asthma. Nature 402, B12-17 (1999).
11. Martinez, F. D. et al. Asthma and wheezing in the first six years of life. The Group Health Medical Associates. N Engl J Med 332, 133-8 (1995).
12. Burrows, B., Martinez, F. D., Halonen, M., Barbee, R. A. & Cline, M. G. Association of asthma with serum IgE levels and skin-test reactivity to allergens. N Engl J Med 320, 271-7 (1989).
13. Illi, S. et al. The pattern of atopic sensitization is associated with the development of asthma in childhood. J Allergy Clin Immunol 108, 709-14 (2001).
14. Akbari, O. et al. CD4+ invariant T-cell-receptor+ natural killer T cells in bronchial asthma. N Engl J Med 354, 1117-29 (2006).
15. Akbari, O. et al. Essential role of NKT cells producing IL-4 and IL-13 in the development of allergen-induced airway hyperreactivity. Nat Med 9, 582-8 (2003).
16. Lisbonne, M. et al. Cutting edge: invariant V alpha 14 NKT cells are required for allergen-induced airway inflammation and hyperreactivity in an experimental asthma model. J Immunol 171, 1637-41 (2003).
17. Sen, Y. et al. V{alpha}24-Invariant NKT Cells from Patients with Allergic Asthma Express CCR9 at High Frequency and Induce Th2 Bias of CD3+ T Cells upon CD226 Engagement. J Immunol 175, 4914-26 (2005).
18. Hamzaoui, A. et al. NKT cells in the induced sputum of severe asthmatics. Mediators Inflamm 2006, 71214 (2006).
19. Bendelac, A., Rivera, M. N., Park, S. H. & Roark, J. H. Mouse CD1-specific NK1 T cells: development, specificity, and function. Annu Rev Immunol 15, 535-62 (1997).
20. Kronenberg, M. & Gapin, L. The unconventional lifestyle of NKT cells. Nat Rev Immunol 2, 557-68 (2002).
21. Brossay, L. et al. CD1d-mediated recognition of an alpha-galactosylceramide by natural killer T cells is highly conserved through mammalian evolution. J Exp Med 188, 1521-8 (1998).
22. Yoshimoto, T. & Paul, W. E. CD4+, NK1.1+ T cells promptly produce interleukin 4 in response to in vivo challenge with anti-CD3. J. Exp. Med. 179, 1285-95 (1994).
23. Cui, J. et al. Inhibition of T helper cell type 2 cell differentiation and immunoglobulin E response by ligand-activated Valpha14 natural killer T cells. J Exp Med 190, 783-92 (1999).
24. Wilson, S. B. et al. Extreme Th1 bias of invariant V□24J□Q T cells in type 1 diabetes. Nature 391, 177-81 (1998).
25. Brigl, M. & Brenner, M. B. CD1: antigen presentation and T cell function. Annu Rev Immunol 22, 817-90 (2004).
26. Cui, J. et al. Requirement for Valpha14 NKT cells in IL-12-mediated rejection of tumors. Science 278, 1623-6 (1997).
27. Busse, W. W. & Lemanske, R. F., Jr. Asthma. N Engl J Med 344, 350-62 (2001).
28. Thomas, S. Y., Lilly, C. M. & Luster, A. D. Invariant natural killer T cells in bronchial asthma. N Engl J Med 354, 2613-6; author reply 2613-6 (2006).
29. Meyer, E. H. et al. Glycolipid activation of invariant T cell receptor+NK T cells is sufficient to induce airway hyperreactivity independent of conventional CD4+ T cells. Proc Natl Acad Sci USA 103, 2782-2787 (2006).
30. Campos, R. A. et al. Cutaneous immunization rapidly activates liver invariant Valpha14 NKT cells stimulating B-1 B cells to initiate T cell recruitment for elicitation of contact sensitivity. J Exp Med 198, 1785-96 (2003).
31. Gumperz, J. E. et al. Murine CD1d-restricted T cell recognition of cellular lipids. Immunity 12, 211-21 (2000).
32. Sadzuka, Y., Kishi, K., Hirota, S. & Sonobe, T. Effect of polyethyleneglycol (PEG) chain on cell uptake of PEG-modified liposomes. J Liposome Res 13, 157-72 (2003).
33. Suzuki, Y. et al. Liposome-encapsulated CpG oligodeoxynucleotides as a potent adjuvant for inducing type 1 innate immunity. Cancer Res 64, 8754-60 (2004).
34. Agea, E. et al. Human CD1-restricted T cell recognition of lipids from pollens. J Exp Med 202, 295-308 (2005).
35. Arreaza, G. A. et al. Neonatal activation of CD28 signaling overcomes T cell anergy and prevents autoimmune diabetes by an IL-4-dependent mechanism. J Clin Invest 100, 2243-53 (1997).
36. Martin, T. R., Gerard, N. P., Galli, S. J. & Drazen, J. M. Pulmonary responses to bronchoconstrictor agonists in the mouse. J. Appl. Physiol. 64, 2318-23 (1988).
37. Akbari, O., R. H. DeKruyff, and D. T. Umetsu. Pulmonary dendritic cells secreting IL-10 mediate T cell tolerance induced by respiratory exposure to antigen. Nature Immunol. 2:725-731 (2001).

All references described herein and throughout the specification are herein incorporated by reference in their entirety.

We claim:

1. A method for treatment and/or prevention of airway hyperreactivity in an individual in need thereof comprising administering to the individual a composition comprising
(a) an agent that attenuates CD1d-restricted NK T cells responses, wherein the agent is selected from the group consisting of antagonists that bind CD1d and inhibit activation of CD1d-restricted NK T cells, agents that block CD1d-specific receptors and CD1d decoys and mimics; and
(b) a pharmaceutically acceptable carrier or diluent.

2. The method of claim 1, wherein the composition is administered in a pharmaceutically acceptable carrier for inhalable administration.

3. The method of claim 1, wherein the agent is a phospholipid that binds CD1d without activating NK T cells.

4. The method of claim 3, wherein the phospholipid is selected from the group consisting of 1,2-Dipalmitoyl-sn-Glycero-3-Phosphoethanolamine (DPPE), 1,2-Dipalmitoyl-sn-Glycero-3-Phosphoethanolamine-N-[Poly(ethylene glycol) 2000] (DPPE-PEG), and phophotidyl inositol.

5. The method of claim 1, wherein the agent is a glycolipid.

6. The method of claim 5, wherein the glycolipid is selected from the group consisting of ceramide, αMan Cer, and βGal Cer.

7. The method of claim 1, wherein the airway hyperreactivity is associated with asthma.

8. The method of claim 1, wherein the composition is administered systemically.

9. A method of preventing or reducing a CD1d-restricted NK T cells responses associated in an airway hyperreactivity, comprising administering to an individual in need thereof prior to exposure to an irritant a composition that comprises an agent that attenuates CD1d-restricted NK T cells responses, wherein the agent is selected from the group consisting of antagonists that bind CD1d and inhibit activation of CD1d-restricted NK T cells, agents that block CD1d-specific receptors on NK T cells, and decoys and mimics.

10. The method of claim 9, wherein the irritant is selected from the group consisting of poison ivy, poison oak, poison sumac, insecticides, dye intermediates, resins, detergents, antioxidants, rubber, latex, metals, fragrances, chemicals, cosmetics, textiles, plastics, pollen, therapeutic agents, skin cleansers, industrial cleaning agents, alkalis, acids, oils, organic solvents, oxidizing agents, reducing agents, plant matter, and animal matter.

11. The method of claim 9, wherein the composition is administered by inhalable system.

12. The method of claim 9, wherein the composition is administered by systemic administration.
